(19) United States
(12) Patent Application Publication
(10) Pub. No.: US 2024/0325045 A1
(43) Pub. Date: Oct. 3, 2024

(54) ELONGATED MEDICAL BODY

(71) Applicant: TERUMO KABUSHIKI KAISHA, Tokyo (JP)

(72) Inventors: Yuya OTAKE, Shizuoka (JP); Tetsuya FUKUOKA, Kanagawa (JP)

(73) Assignee: TERUMO KABUSHIKI KAISHA, Tokyo (JP)

(21) Appl. No.: 18/609,038

(22) Filed: Mar. 19, 2024

(30) Foreign Application Priority Data
Mar. 29, 2023 (JP) 2023-053226

(51) Int. Cl. A61B 17/34; A61M 25/00; A61M 25/01; A61M 25/06; A61M 25/09; A61M 25/10

(52) U.S. Cl. CPC A61B 17/3415; A61B 2017/3433; A61M 25/0029; A61M 2025/0183; A61M 2025/0681; A61M 25/09; A61M 25/104

(57) ABSTRACT

An elongated medical body includes a main body portion extending in an axial direction and including a guide wire lumen through which a guide wire can pass, a shaft portion extending in the axial direction and connected to a proximal end side of the main body portion, and a guide wire port which is provided at a connection point between the main body portion and the shaft portion, and through which the guide wire can enter and exit, and an inner diameter of the guide wire lumen is equal to or larger than an outer diameter of the shaft portion.

### CROSS-REFERENCES TO RELATED APPLICATIONS

This application claims priority to Japanese Patent Application No. 2023-053226 filed on Mar. 29, 2023, the entire content of which is incorporated herein by reference.

### TECHNOLOGICAL FIELD

The present invention generally relates to an elongated medical body.

### BACKGROUND DISCUSSION

A guiding catheter, which is one type of catheter, is a catheter to be used when a balloon catheter, or the like, for treating a coronary artery (hereinafter, coronary artery) of a heart, a lower limb artery, or the like, is inserted to a target site.

In particular, in a guiding catheter to be used for intravascular treatment of a coronary artery, a lower limb artery, or the like, a diameter of a blood vessel to be treated is large, and thus, it is preferable to increase an inner diameter of the guiding catheter and reduce a wall thickness of the guiding catheter in order to allow selection of a treatment device having a large outer diameter or sending more treatment devices to a distal end at the same time so as to allow delivery of a plurality of treatment devices to the treatment target site at the same time.

In a case where the wall thickness of the catheter is thin as described above, a configuration in which an inner catheter or a dilator (collectively referred to as an elongated medical body) is disposed in a lumen of the catheter is adopted for the purpose of preventing occurrence of kinking of the catheter and lowering of pushability.

The elongated body as described above is required to have pushability with respect to a lesion, and thus, an over-the-wire (OTW) type method has been often used. However, in the over-the-wire type method, in order to remove the elongated medical body, for example, if a total length of the guiding catheter is 150 cm, a total length of the elongated medical body arranged such that a distal end protrudes into the lumen of the guiding catheter is required to be longer than the total length of the guiding catheter, and a guide wire of twice or more the total length of the elongated medical body, for example, about 400 cm is required, which makes operation at the time of removal very complicated. In addition, in the over-the-wire type method, it is also difficult to replace a guide wire having high rigidity, for example, a 0.035 inch guide wire.

From the above, in recent years, instead of the over-the-wire type method, a rapid exchange (RX) type method in which devices can be easily replaced has been disclosed. An example is Japanese Patent Publication No. 5221032 (JP 5221032 B).

### SUMMARY

In a rapid exchange type balloon catheter to be used for general percutaneous coronary intervention (PCI), a guide wire of 0.014 inches (0.36 mm) is mainly used, and a shaft of a rapid exchange type catheter device is generally thicker than the guide wire.

On the other hand, particularly in a catheter for treating a lower limb artery, a 0.035 inch (0.89 mm) guide wire is used. The present inventors have found a problem that in a case where a thicker shaft portion is employed for a 0.035 inch guide wire, a guide wire lumen and the shaft portion become thick, which makes insertion into a guiding catheter difficult.

The elongated medical body that is easy to be inserted into a guiding catheter even in a case where a relatively thick guide wire (for example, 0.035 inches) is used in a rapid exchange type elongated medical body.

(1) The elongated medical body is disposed so as to have a distal end protruding into a lumen of a catheter and assists insertion of the catheter. The elongated medical body includes: a main body portion extending in an axial direction and including a guide wire lumen through which a guide wire can pass; a shaft portion extending in the axial direction and connected to a proximal end side of the main body portion; and a guide wire port which is provided at a connection portion between the main body portion and the shaft portion, and through which the guide wire can enter and exit, in which an inner diameter of the guide wire lumen is equal to or larger than an outer diameter of the shaft portion.

(2) The elongated medical body according to (1) in which the outer diameter of the shaft portion is equal to or greater than 0.4 mm, and the inner diameter of the guide wire lumen is equal to or greater than 0.9 mm.

(3) The elongated medical body according to (1) or (2), in which the outer diameter of the shaft portion is equal to or greater than 0.6 mm.

(4) The elongated medical body according to any one of (1) to (3), in which a cross-sectional area of the shaft portion is smaller than a cross-sectional area of the guide wire lumen.

(5) The elongated medical body according to any one of (1) to (3), in which three shaft portions are provided.

(6) The elongated medical body according to (5), in which a cross-sectional area of the shaft portions is equal to or larger than a cross-sectional area of the guide wire lumen.

According to the elongated medical body configured as described above, the inner diameter of the guide wire lumen is equal to or larger than the outer diameter of the shaft portion, so that the outer diameter of the shaft portion can be reduced, and the outer diameter of the entire elongated medical body can be reduced. It is therefore possible to provide an elongated medical body that is easily inserted into a guiding catheter even in a case where a relatively thick guide wire (for example, 0.035 inches) is used in a rapid exchange type elongated medical body.

Another aspect of the disclosure involves an elongated medical body usable in combination with a catheter provided with a lumen so that a distal end of the elongated medical body is positionable in the lumen of the catheter to assist insertion of the catheter into a living body. The elongated medical body comprises a main body portion extending in an axial direction between a proximal end of the main body portion and a distal end of the main body portion, with the main body portion including a proximal portion that terminates at the proximal end of the main body portion, the proximal portion of the main body portion including a recess. The main body portion includes a guide wire lumen extending axially throughout the main body portion from the proximal end of the main body portion to the distal end of the main body portion, with the guide wire lumen being open to outside the main body portion at both the proximal end of the main body portion and the distal end of the main body so that a guide wire is positionable in the guide wire lumen and extends distally beyond the distal end of the main body and proximally beyond the proximal end of the main body portion. A shaft portion extends in the axial direction and incudes a distal portion positioned in the recess in the main body portion and fixed to the main body portion so that the shaft portion extends away from the main body portion. The elongated medical body also includes a guide wire port through which the guide wire enters and exits the guide wire lumen, the guide wire port being provided at the connection portion between the main body portion and the shaft portion. The inner diameter of the guide wire lumen is equal to or larger than the outer diameter of the shaft portion.

Another aspect of the disclosure involves an elongated medical body in combination with a guide wire. The elongated medical body is usable together with a catheter having a lumen so that a distal end of the elongated medical body is positionable in the lumen of the catheter to assist insertion of the catheter into a living body. The guide wire is an elongated guide wire that has an outer diameter equal to or greater than 0.035 in. The elongated medical body comprises a main body portion extending in an axial direction between a proximal end of the main body portion and a distal end of the main body portion, with the main body portion including a guide wire lumen extending axially throughout the main body portion from the proximal end of the main body portion to the distal end of the main body portion, and the guide wire lumen being open to outside the main body portion at both the proximal end of the main body portion and the distal end of the main body so that the guide wire is positionable in the guide wire lumen while also extending distally beyond the distal end of the main body and proximally beyond the proximal end of the main body portion. A shaft portion extends in the axial direction and includes a shaft portion having a distal portion fixed to main body portion at a connection portion while an adjacent part of the shaft portion extends away from the main body portion. The elongated medical body also includes a guide wire port through which the guide wire enters and exits the guide wire lumen in the main body portion, wherein the guide wire port is provided at the connection portion between the main body portion and the shaft portion. In addition, the inner diameter of the guide wire lumen is equal to or larger than the outer diameter of the shaft portion.

### BRIEF DESCRIPTION OF THE DRAWINGS

FIG. 1 is an overall view illustrating an elongated medical body according to an embodiment disclosed here.

FIG. 2 is an overall view illustrating a guiding catheter into which the elongated medical body according to the present embodiment is to be inserted.

FIG. 3 is an enlarged front cross-sectional view along a long axis or longitudinal central axis illustrating the vicinity of a distal end in a state where the elongated medical body according to the present embodiment is inserted into the guiding catheter.

FIG. 4 is a cross-sectional view taken along the section line 4-4 in FIG. 3.

FIG. 5 is an enlarged plan view illustrating the vicinity of a distal end of the elongated medical body.

FIG. 6 is an enlarged front cross-sectional view illustrating the vicinity of the distal end of the elongated medical body along a long axis or longitudinal central and illustrates a state where a guide wire is inserted.

FIG. 7 is a cross-sectional view taken along the section line 7-7 in FIG. 6.

FIG. 8 is a cross-sectional view taken along the section line 8-8 of FIG. 6.

FIG. 9 is a cross-sectional view taken along the section line 9-9 of FIG. 6.

FIG. 10 is a view corresponding to FIG. 5 of an elongated medical body according to a first modification.

FIG. 11 is a view corresponding to FIG. 6 of the elongated medical body according to the first modification.

FIG. 12 is a cross-sectional view taken along the section line 12-12 in FIG. 11.

FIG. 13 is a cross-sectional view taken along the section line 13-13 in FIG. 11.

FIG. 14 is a view corresponding to FIG. 5 of an elongated medical body according to a second modification.

FIG. 15 is a view corresponding to FIG. 6 of the elongated medical body according to the second modification.

FIG. 16 is a cross-sectional view taken along the section line 16-16 in FIG. 15.

FIG. 17 is a cross-sectional view taken along the section line 17-17 in FIG. 15.

FIG. 18 is a view corresponding to FIG. 5 of an elongated medical body according to a third modification.

FIG. 19 is a view corresponding to FIG. 6 of the elongated medical body according to the third modification.

FIG. 20 is a cross-sectional view taken along the section line 20-20 in FIG. 19.

FIG. 21 is a cross-sectional view taken along the section line 21-21 in FIG. 19.

FIG. 22 is a cross-sectional view taken along the section line 22-22 in FIG. 19.

FIG. 23 is a cross-sectional view taken along the section line 23-23 of FIG. 19.

FIG. 24 is a cross-sectional view taken along the section line 24-24 of FIG. 19.

### DETAILED DESCRIPTION

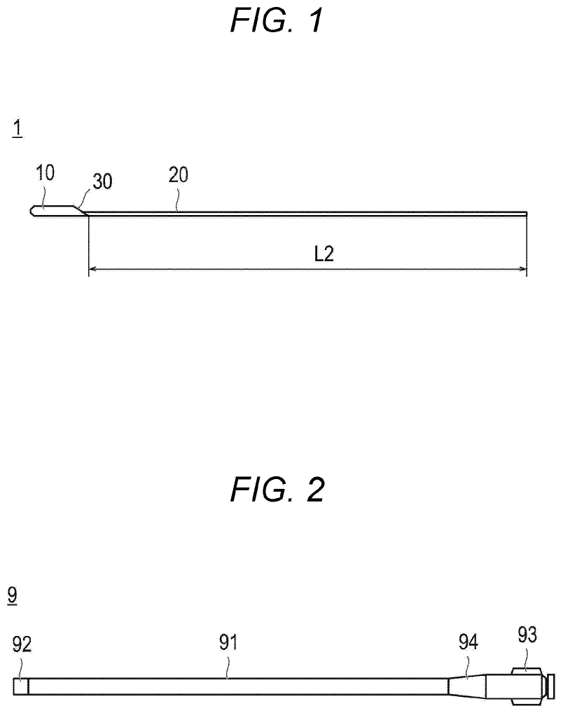

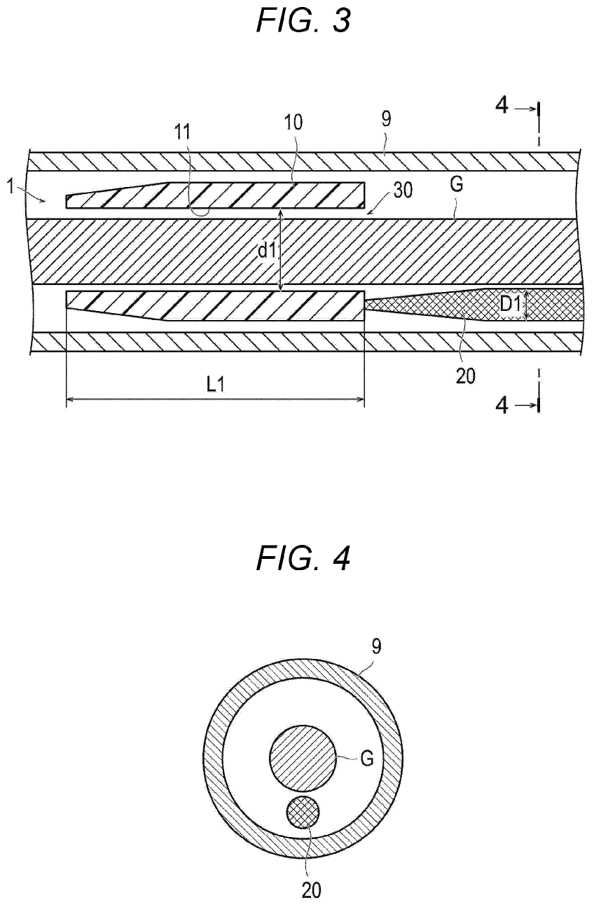

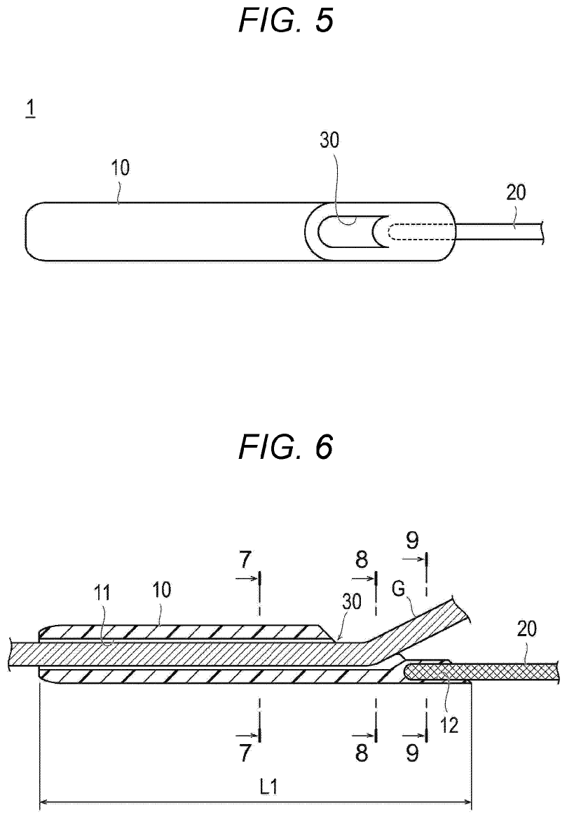

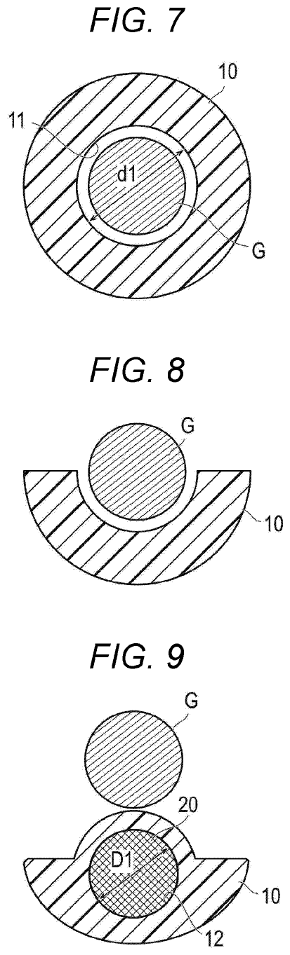

Hereinafter, an elongated medical body 1 and a catheter 9 into which the elongated medical body 1 is to be inserted according to an embodiment disclosed here will be described with reference to FIGS. 1 to 9. FIG. 1 is an overall view illustrating the elongated medical body 1 according to an embodiment that represent one example of the new elongated medical body. FIG. 2 is an overall view illustrating the catheter 9 into which the elongated medical body 1 according to the present embodiment is to be inserted. FIG. 3 is an enlarged front view along a long axis illustrating the vicinity of a distal end in a state where the elongated medical body 1 according to the present embodiment is inserted into the catheter 9. FIG. 4 is a cross-sectional view taken along line 4-4 in FIG. 3. FIG. 5 is an enlarged plan view illustrating the vicinity of a distal end of the elongated medical body 1. FIG. 6 is an enlarged front cross-sectional view of the vicinity of the distal end of the elongated medical body 1 along the long axis and illustrates a state in which a guide wire G is inserted. FIG. 7 is a cross-sectional view taken along the section line 7-7 of FIG. 6. FIG. 8 is a cross-sectional view taken along the section line 8-8 in FIG. 6. FIG. 9 is a cross-sectional view taken along the section line 9-9 of FIG. 6.

The catheter 9 illustrated in FIG. 2 is used as a guiding catheter for guiding a treatment catheter (device) such as an expansion catheter (balloon catheter) for PTCA or a catheter (stent conveyance catheter) for conveying a stent in a reduced diameter state to a stenosed part and expanding and maintaining the stenosed part by expanding the diameter at the stenosed part, to a target site such as a stenosed part of a coronary artery or a lower limb artery.

As illustrated in FIG. 2, the catheter 9 includes a tube 91, a flexible distal end tip 92 provided on a distal end side of the tube 91, a hub 93 provided on a proximal end side of the tube 91, and a kink protector 94 provided on a distal end side of the hub 93. The catheter 9 has a known configuration, and thus, a detailed description thereof will be omitted.

The elongated medical body 1 illustrated in FIG. 1 is a medical instrument that is disposed so that its distal end protrudes into a lumen of the catheter 9 and assists insertion of the catheter 9. In other words, the elongated medical body 1 is used as an inner catheter or a dilator.

As illustrated in FIGS. 1 to 6, the elongated medical body 1 includes a main body portion 10 including a guide wire lumen 11 through which the guide wire G can be inserted, and a shaft portion 20 connected to a proximal end side of the main body portion 10. The elongated medical body 1 is configured as a so-called rapid exchange type elongated medical body in which a guide wire port 30 through which the guide wire G can enter and exit is formed at a connection portion between the main body portion 10 and the shaft portion 20.

By configuring the elongated medical body 1 as a rapid exchange type in this manner, it is possible to improve efficiency of the procedure. In particular, by using the elongated medical body 1, an operator can quickly and efficiently perform operation of removing the guide wire G used to penetrate the lesion from the elongated medical body 1. Further, the 0.035 inch guide wire G having high rigidity can be replaced quickly and efficiently.

As illustrated in FIGS. 1, 3, and 5, the main body portion 10 extends in the axial direction. As illustrated in FIGS. 3, 6, and 7, the main body portion 10 includes a guide wire lumen 11 through which the guide wire G can pass and a recessed portion 12 into which the shaft portion 20 (distal portion of the shaft portion) is to be inserted (is inserted).

The distal end of the main body portion 10 preferably has a tapered shape. According to this configuration, it is easy to insert the elongated medical body 1 inserted into the catheter 9 into the living body.

A length L1 (see FIG. 6) along the axial direction of the main body portion 10 is not particularly limited, but is 50 to 900 mm, and preferably 100 mm to 600 mm. An inner diameter d1 (see FIG. 7) of the guide wire lumen 11 of the main body portion 10 is not particularly limited, but is 0.9 to 1.3 mm.

As a material constituting the main body portion 10, polyethylene, polypropylene, polyurethane, a polyurethane elastomer, polyamide, a polyamide elastomer, polyester, a polyester elastomer, silicone rubber, natural rubber, or the like, may be used, a blend or a multilayer tube having an inner layer and an outer layer may be used, or a metal wire such as SUS, tungsten, or NiTi or a resin wire may be embedded as a reinforcing body such as a coil or a braid.

As illustrated in FIGS. 1, 5, and 6, the shaft portion 20 extends in the axial direction. As illustrated in FIG. 6, a distal end of the shaft portion 20 is fixed by an adhesive or fusion while being inserted into the recessed portion 12 of the main body portion 10. A method of fixing the shaft portion 20 to the main body portion 10 is not particularly limited. The distal end of the shaft portion 20 is formed in a round shape.

A length L2 (see FIG. 1) along the axial direction of the shaft portion 20 is not particularly limited as long as a proximal end of the shaft portion 20 protrudes from a proximal end opening of the hub 93 to a proximal end side when the distal end is arranged to protrude into the lumen of the catheter, and is, for example, 600 to 2000 mm, preferably 1000 mm to 1500 mm. An outer diameter D1 (see FIG. 9) of the shaft portion 20 is not particularly limited as long as it is smaller than an inner diameter of the guide wire lumen into which the extremely thick guide wire G (for example, a guide wire having an outer diameter equal to or greater than 0.035 inches) can be inserted, but is preferably 0.4 to 1.5 mm and more preferably 0.6 to 0.8 mm.

As described above, an inner diameter d1 of the guide wire lumen 11 is equal to or larger than the outer diameter D1 of the shaft portion 20. In the present embodiment, the shaft portion 20 includes one shaft portion. Thus, a cross-sectional area of the shaft portion 20 is equal to or smaller than a cross-sectional area of the guide wire lumen 11. According to this configuration, the outer diameter of the shaft portion 20 can be reduced, and the outer diameter of the entire elongated medical body 1 can be reduced. Thus, even in a case where a relatively thick guide wire G (for example, 0.035 inches) is used in the rapid exchange type elongated medical body 1, insertion into the catheter 9 is facilitated.

As a material constituting the shaft portion 20, a metal such as stainless steel or Ni—Ti may be used. At least part of the shaft portion 20 may be coated by a fluorine-based resin such as polytetrafluoroethylene (PTFE), ethylene-tetrafluoroethylene copolymer (ETFE), or perfluoroalkoxy fluorine resin (PFA), an olefin-based resin such as polyethylene or polypropylene or a mixture thereof, polyurethane, polyester, polyamide, polyamide elastomer, a mixture of the olefin-based resin and ethylene-vinyl acetate copolymer, a silicone resin, or the like.

As illustrated in FIGS. 5, 6, and 8, the guide wire port 30 is configured to be inclined (i.e., the guide wire port 30 is inclined). According to this configuration, the guide wire G can be easily inserted into the elongated medical body 1. FIG. 6 shows that the guide wire port 30 is configured so that a proximal portion of the guide wire lumen 11 (i.e., a portion proximal of the lead line for reference numeral “30”) is open in the upward direction as depicted by the cross-section in FIG. 8. Stated differently, FIG. 7 shows the curved inner surface that completely surrounds the guide wire lumen 11 for a 360° circumferential extent, and FIG. 8 shows that in the proximal portion of the main body portion 10 (i.e., at the guide wire port 30 and adjacent the connection portion at which the shaft portion 20 is connected to the main body portion 10), the curved inner surface continues along the lower part of the main body portion 10 while the upper part of the main body portion 10 is open (the curved inner surface in the proximal portion of the main body portion 10 has a circumferential extent less than) 360°.

### <Elongated Medical Body 2 According to First Modification>

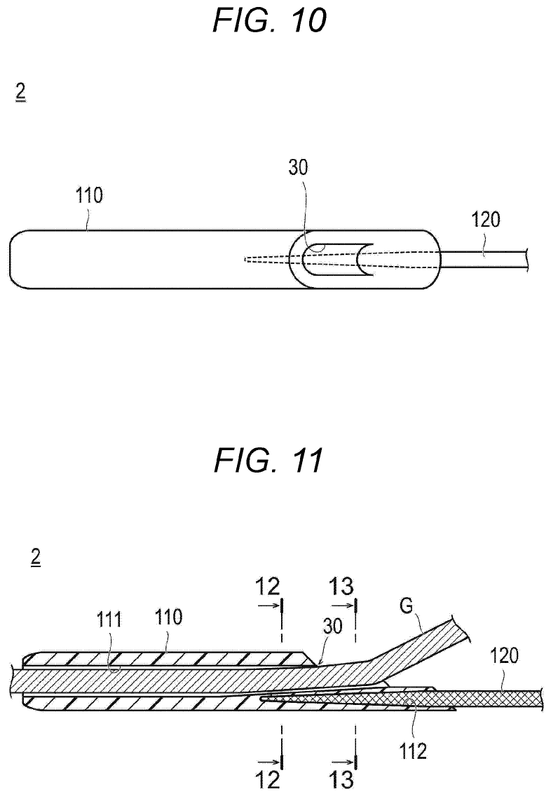

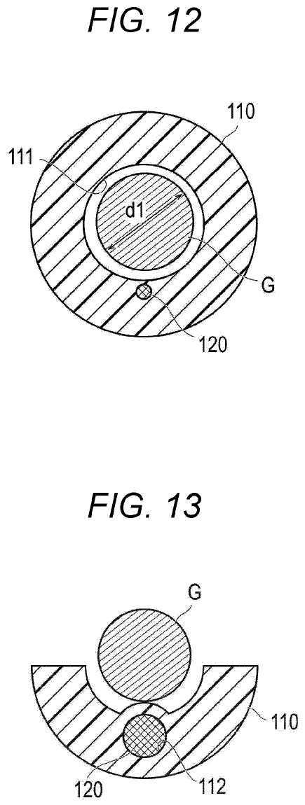

Next, a configuration of an elongated medical body 2 according to a first modification will be described with reference to FIGS. 10 to 13. FIG. 10 is a view corresponding to FIG. 5 of the elongated medical body 2 according to the first modification. FIG. 11 is a view corresponding to FIG. 6 of the elongated medical body 2 according to the first modification. FIG. 12 is a cross-sectional view taken along the section line 12-12 in FIG. 11. FIG. 13 is a cross-sectional view taken along the section line 13-13 of FIG. 11.

The elongated medical body 2 according to the first modification is different from the elongated medical body 1 according to the above-described embodiment in a shape of the distal end, and the like, of the shaft portion 120. The same components as those of the elongated medical body 1 according to the above embodiment are denoted by the same reference numerals, and the description thereof will be omitted.

As illustrated in FIGS. 10 to 13, the elongated medical body 2 according to the first modification includes a main body portion 110 including a guide wire lumen 111 through which a guide wire G can be inserted, and a shaft portion 120 connected to a proximal end side of the main body portion 110. The elongated medical body 2 is configured as a so-called rapid exchange type elongated medical body in which a guide wire port 30 through which the guide wire G can enter and exit is formed at a connection portion between the main body portion 110 and the shaft portion 120.

As illustrated in FIGS. 11, 12, and 13, the main body portion 110 includes the guide wire lumen 111 through which the guide wire G can pass and a recessed portion 112 into which the shaft portion 120 is to be inserted (is inserted).

As illustrated in FIG. 11, a distal end of the shaft portion 120 is fixed by an adhesive or fusion while being inserted into the recessed portion 112 of the main body portion 110. As illustrated in FIG. 11, the distal end of the shaft portion 120 has a tapered shape tapered toward the distal end. According to this configuration, a rapid change in physical properties in the vicinity of the distal end of the shaft portion 120 can be reduced, so that kink resistance can be improved.

### <Elongated Medical Body 3 According to Second Modification>

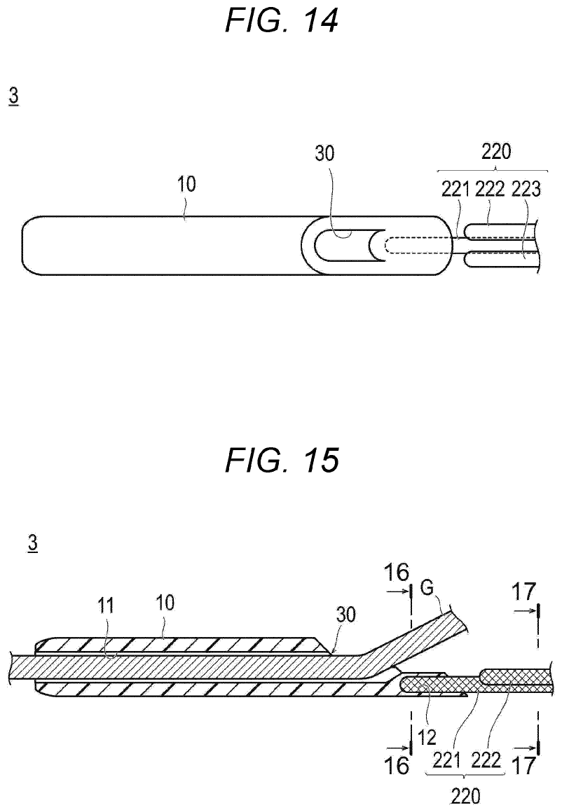

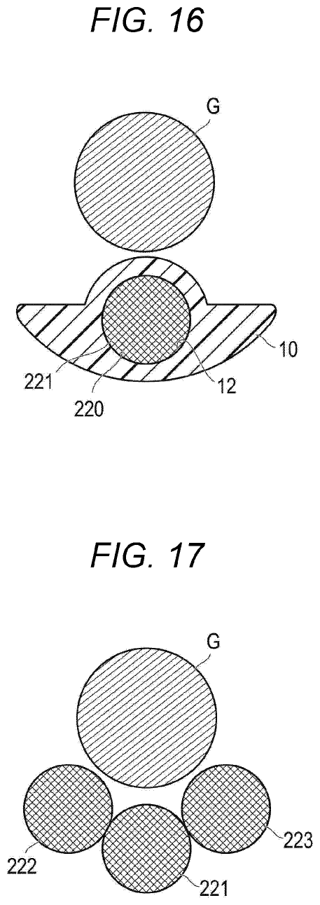

Next, a configuration of an elongated medical body 3 according to a second modification will be described with reference to FIGS. 14 to 17. FIG. 14 is a view corresponding to FIG. 5 of the elongated medical body 3 according to the second modification. FIG. 15 is a view corresponding to FIG. 6 of the elongated medical body 3 according to the second modification. FIG. 16 is a cross-sectional view taken along the section line 16-16 of FIG. 15. FIG. 17 is a cross-sectional view taken along the section line 17-17 of FIG. 15.

The elongated medical body 3 according to the second modification is different from the elongated medical body 1 according to the above-described embodiment in the number of shaft portions 220. The same components as those of the elongated medical body 1 according to the above embodiment are denoted by the same reference numerals, and a detailed description thereof is not repeated.

As illustrated in FIGS. 14 to 17, the elongated medical body 3 according to the second modification includes a main body portion 10 including a guide wire lumen 11 through which a guide wire G can be inserted, and a shaft portion 220 connected to a proximal end side of the main body portion 10. The elongated medical body 3 is configured as a so-called rapid exchange type elongated medical body in which a guide wire port 30 through which the guide wire G can enter and exit is formed at a connection portion between the main body portion 10 and the shaft portion 220. The main body portion 10 of the elongated medical body 3 according to the second modification has the same configuration as the configuration of the main body portion 10 of the elongated medical body 1 according to the above-described embodiment, and thus, a detailed description thereof will not be repeated.

As illustrated in FIGS. 14 to 17, the shaft portion 220 includes three shaft portions 221, 222, and 223. The shaft portion 220 may be configured by twisting the three shaft portions 221, 222, and 223 to each other or around each other, or may be configured by bonding the three shaft portions 221, 222, and 223 to each other with an adhesive. In the second modification, all three shaft portions 221, 222, and 223 have the same thickness, for example, a diameter of 0.6 mm.

As illustrated in FIGS. 14 and 17, the first shaft portion 221 is disposed between the second shaft portion 222 and the third shaft portion 223 and below the second shaft portion 222 and the third shaft portion 223. According to this configuration, as illustrated in FIG. 17, the shaft portion 220 has a recessed shape (U-shape), and the guide wire G can be disposed at a portion having the recessed shape, and the guide wire G can be suitably moved forward and backward.

As illustrated in FIG. 15, a distal end of the first shaft portion 221 of the shaft portion 220 is fixed by an adhesive or solder in a state of being inserted into the recessed portion 12 of the main body portion 10.

Also in the second modification, the inner diameter d1 of the guide wire lumen 11 is equal to or larger than the outer diameter D1 of one first shaft portion 221 (or the second shaft portion 222, the third shaft portion 223). In the elongated medical body 3 according to the second modification, the three shaft portions 220 are provided, and thus, a total cross-sectional area of the three shaft portions 220 is equal to or larger than a cross-sectional area of the guide wire lumen 11. According to the elongated medical body 3 configured as described above, the shaft portion 220 can be made thicker than the shaft portion 20 of the elongated medical body 1 according to the above-described embodiment, so that rigidity of the shaft portion 220 is increased, and pushability of the elongated medical body 3 can be improved.

### <Elongated Medical Body 4 According to Third Modification>

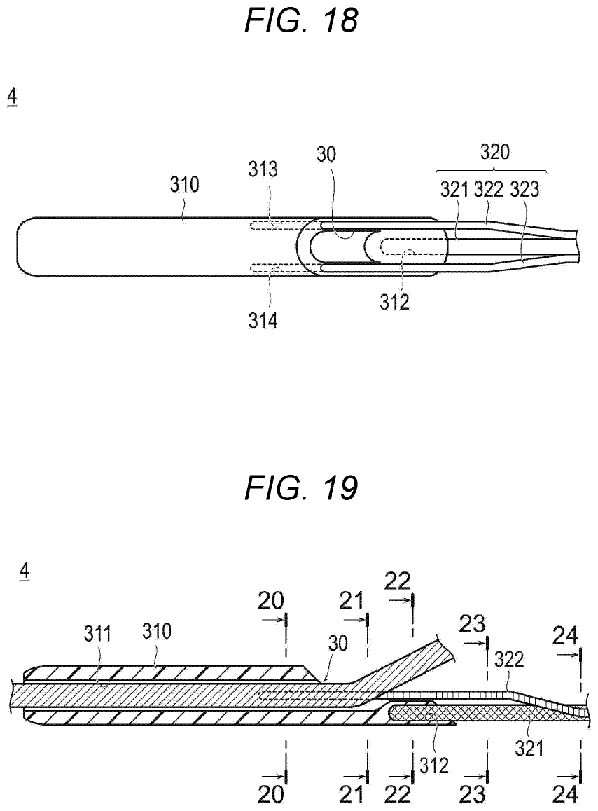

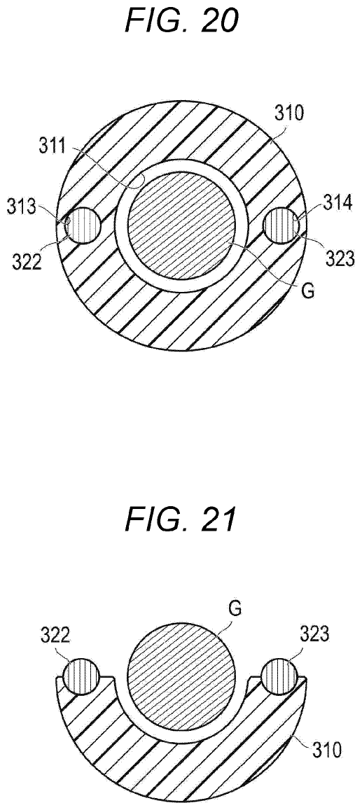

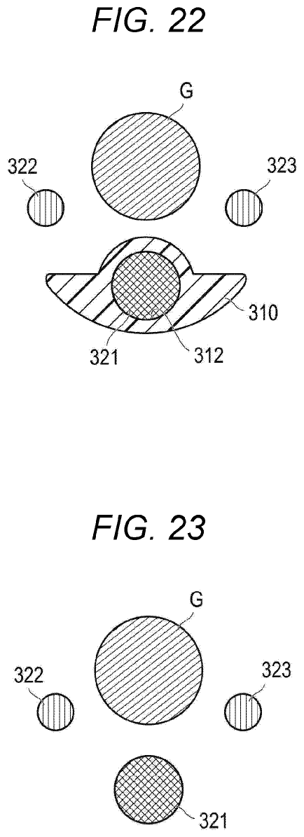

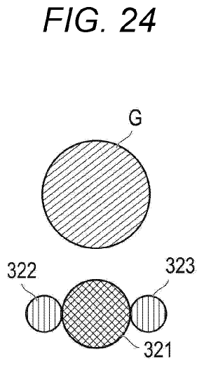

Next, a configuration of an elongated medical body 4 according to a third modification will be described with reference to FIGS. 18 to 24. FIG. 18 is a view corresponding to FIG. 5 of the elongated medical body 4 according to the third modification. FIG. 19 is a view corresponding to FIG. 6 of the elongated medical body 4 according to the third modification. FIG. 20 is a cross-sectional view taken along the section line 20-20 of FIG. 19. FIG. 21 is a cross-sectional view taken along the section line 21-21 of FIG. 19. FIG. 22 is a cross-sectional view taken along the section line 22-22 of FIG. 19. FIG. 23 is a cross-sectional view taken along line 23-23 of FIG. 19. FIG. 24 is a cross-sectional view taken along the section line 24-24 of FIG. 19.

The elongated medical body 4 according to the third modification is different from the elongated medical body 1 according to the above-described embodiment in the configuration of the shaft portion 320. The same components as those of the elongated medical body 1 according to the above embodiment are denoted by the same reference numerals, and a detailed description thereof will not be repeated.

As illustrated in FIGS. 18 to 24, the elongated medical body 4 according to the third modification includes a main body portion 310 including a guide wire lumen 311 through which a guide wire G can be inserted, and a shaft portion 320 connected to a proximal end side of the main body portion 310. The elongated medical body 4 is configured as a so-called rapid exchange type elongated medical body in which a guide wire port 30 through which the guide wire G can enter and exit is formed at a connection portion between the main body portion 310 and the shaft portion 320.

As illustrated in FIGS. 18 to 22, the main body portion 310 includes the guide wire lumen 311 through which the guide wire G can pass, a first recessed portion 312 into which the first shaft portion 321 of the shaft portion 320 is to be inserted (is inserted), a second recessed portion 313 into which the second shaft portion 322 of the shaft portion 320 is to be inserted (is inserted), and a third recessed portion 314 into which the third shaft portion 323 of the shaft portion 320 is to be inserted (is inserted).

As illustrated in FIG. 18, the second recessed portion 313 and the third recessed portion 314 extend up to a distal end side of the first recessed portion 312. That is, the distal end portion of the second and third recessed portions 313, 314 extend and are located distally beyond the distal end of the first recessed portion 312. As illustrated in FIGS. 20 and 22, the second recessed portion 313 and the third recessed portion 314 are formed (located) above the first recessed portion 312. As illustrated in FIG. 18, the second recessed portion 313 and the third recessed portion 314 are thinner than the first recessed portion 312. For example, as shown in FIGS. 21-23, the second and third recessed portions 313, 314 have a smaller outer diameter (and smaller cross-sectional area) than the outer diameter (cross-sectional area) of the first recessed portion 312.

As illustrated in FIGS. 18 to 24, the shaft portion 320 includes three shaft portions 321, 322, and 323. The shaft portion 320 may be configured by twisting the three shaft portions 321, 322, and 323 to each other or around each other, or may be configured by bonding the three shaft portions 321, 322, and 323 to each other with an adhesive or solder. In the third modification, the first shaft portion 321 is thicker than the second shaft portion 322 and the third shaft portion 323, for example, has a diameter of 0.6 mm, and the second shaft portion 322 and the third shaft portion 323 collectively have substantially the same thickness, for example, a diameter of 0.3 mm.

As illustrated in FIG. 24, the first shaft portion 321 is disposed at the same height as the second shaft portion 322 and the third shaft portion 323 while being in contact with the second shaft portion 322 and the third shaft portion 323 at a position indicated by the section line 24-24 in FIG. 19. That is, as seen in FIG. 19, at a position proximal of the distal end of the first shaft portion 321, the first shaft portion 321 is disposed at the same height as the second and third shaft portions 322, 323, but at a position closer to the and at the distal end of the first shaft portion 321, the second and third shaft portions 322, 323 are positioned at elevationally different locations (elevationally higher) than the first shaft portion 321.]

The second shaft portion 322 and the third shaft portion 323 are curved or angled upward and in a width direction (a left-right direction in FIG. 23) from the center between the position indicated by the section line 24-24 and the position indicated by the section line 23-23 in FIG. 19. That is, as shown in the side view of FIG. 19, from the position indicated by the section line 24-24 to the position indicated by the section line 23-23, the second and third shaft portions 322, 323 extend upwardly away from a horizontal plane containing the central axis of the first shaft portion 321. At the same time, the second and third shaft portions 322, 323 diverge laterally away from one another as shown in FIG. 18. On the other hand, the first shaft portion 321 is disposed at the same height along the axial direction, meaning the first shaft portion 321. Thus, as illustrated in FIG. 23, the second shaft portion 322 and the third shaft portion 323 are disposed above the first shaft portion 321 at a position of the section line 23-23 in FIG. 19.

As illustrated in FIG. 19, the distal end of the first shaft portion 321 of the shaft portion 320 is fixed by an adhesive in a state of being inserted into the first recessed portion 312 of the main body portion 310. As illustrated in FIG. 18, the distal end of the second shaft portion 322 of the shaft portion 320 is fixed by an adhesive in a state of being inserted into the second recessed portion 313 of the main body portion 310. As illustrated in FIG. 18, the distal end of the third shaft portion 323 of the shaft portion 320 is fixed by an adhesive in a state of being inserted into the third recessed portion 314 of the main body portion 310.

Also in the third modification, the inner diameter d1 of the guide wire lumen 311 is equal to or larger than the outer diameter D1 of one first shaft portion 321 (or the second shaft portion 322 or the third shaft portion 323). In the elongated medical body 4 according to the third modification, the three shaft portions 320 are provided, and thus, a total cross-sectional area of the three shaft portions 320 is equal to or larger than a cross-sectional area of the guide wire lumen 311. According to the elongated medical body 4 configured in this manner, the shaft portion 320 is made thicker (larger cross-sectional area) than the shaft portion 20 of the elongated medical body 1 according to the above-described embodiment, rigidity of the shaft portion 220 is increased, and a rapid physical property step between the main body portion 310 made of substantially resin and the shaft portion 320 made of substantially metal with respect to a semicircular proximal end opening 330 is alleviated, so that pushability of the elongated medical body 4 can be improved and deflection in a meandering blood vessel can be prevented.

Furthermore, in the elongated medical body 4 according to the third modification, the second shaft portion 322 is fixed to the second recessed portion 313 and the third shaft portion 323 is fixed to the third recessed portion 314, so that buckling of the elongated medical body 4 can be suitably prevented. The shaft portion 320, the distal end of the second shaft portion 322, and the distal end of the third shaft portion 323 are formed in a round shape or a tapered shape.

Although the elongated medical bodies 1, 2, 3, and 4 disclosed here have been described above with reference to the embodiment and modifications, the present invention is not limited to only the configuration described in the embodiment and can be appropriately changed on the basis of the description of the claims.

For example, in the third and fourth modifications described above, three shaft portions are provided, but two or four or more shaft portions may be provided.

The detailed description above describes an elongated medical body and a catheter in combination with an elongated medical body representing examples of the new elongated medical body and catheter/elongated medical body combination disclosed here. The invention is not limited, however, to the precise embodiments, modifications and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. An elongated medical body usable in combination with a catheter provided with a lumen so that a distal end of the elongated medical body is positionable in the lumen of the catheter to assist insertion of the catheter into a living body, the elongated medical body comprising:
a main body portion extending in an axial direction between a proximal end of the main body portion and a distal end of the main body portion, the main body portion including a proximal portion that terminates at the proximal end of the main body portion, the proximal portion of the main body portion including a recess;
the main body portion including a guide wire lumen extending axially throughout the main body portion from the proximal end of the main body portion to the distal end of the main body portion, the guide wire lumen being open to outside the main body portion at both the proximal end of the main body portion and the distal end of the main body so that a guide wire is positionable in the guide wire lumen and extends distally beyond the distal end of the main body and proximally beyond the proximal end of the main body portion, the guide wire lumen possessing an inner diameter;
a shaft portion extending in the axial direction, the shaft portion including a distal portion positioned in the recess in the main body portion and fixed to the main body portion so that the shaft portion extends away from the main body portion, the shaft portion possessing an outer diameter;
a guide wire port through which the guide wire enters and exits the guide wire lumen, the guide wire port being provided at the connection portion between the main body portion and the shaft portion; and
the inner diameter of the guide wire lumen being equal to or larger than the outer diameter of the shaft portion.

2. The elongated medical body according to claim 1, wherein the main body portion includes a curved inner surface surrounding the guide wire lumen and having a 360° circumferential extent, the main body portion at the guide wire port including a continuation of the curved inner surface having a circumferential extent less than 360°.

3. The elongated medical body according to claim 1, wherein the main body portion is made of resin and the shaft portion is made of metal.

4. The elongated medical body according to claim 1, wherein the shaft portion is a first shaft portion, and further comprising a second shaft portion and a third shaft portion that extend along the first shaft portion in the axial direction.

5. The elongated medical body according to claim 4, wherein a total cross-sectional area of the first shaft portion, the second shaft portion and the third shaft portion is equal to or larger than a cross-sectional area of the guide wire lumen.

6. The elongated medical body according to claim 4, wherein portions of the second shaft portion and the third shaft portion diverge away from each other and terminate at distal ends, the distal end of the second shaft portion and the distal end of the third shaft portion being positioned in respective recesses in the main body portion and being fixed to the main body portion.

7. The elongated medical body according to claim 1, wherein the shaft portion is a first shaft portion, and further comprising a second shaft portion positioned adjacent to and extending along the first shaft portion, the second shaft portion having a distal end that is spaced proximally from both the proximal end side of the main body portion and the connection portion.

8. The elongated medical body according to claim 1, wherein the outer diameter of the shaft portion is equal to or greater than 0.4 mm, and the inner diameter of the guide wire lumen is equal to or greater than 0.9 mm.

9. The elongated medical body according to claim 1, wherein a cross-sectional area of the shaft portion is smaller than a cross-sectional area of the guide wire lumen.

10. An elongated medical body usable in combination with a catheter that has a lumen so that a distal end of the elongated medical body protrudes into the lumen of the catheter and assists insertion of the catheter into a living body, the elongated medical body comprising:
a main body portion extending in an axial direction and including a guide wire lumen through which is passable a guide wire, the guide wire lumen possessing an inner diameter;
a shaft portion extending in the axial direction and connected to a proximal end side of the main body portion at a connection portion, the shaft portion possessing an outer diameter;
a guide wire port through which the guide wire enters and exits the guide wire lumen, the guide wire port being provided at the connection portion between the main body portion and the shaft portion; and
the inner diameter of the guide wire lumen being equal to or larger than the outer diameter of the shaft portion.

11. The elongated medical body according to claim 10,
wherein the outer diameter of the shaft portion is equal to or greater than 0.4 mm, and
the inner diameter of the guide wire lumen is equal to or greater than 0.9 mm.

12. The elongated medical body according to claim 10, wherein the outer diameter of the shaft portion is equal to or greater than 0.6 mm.

13. The elongated medical body according to claim 10, wherein a cross-sectional area of the shaft portion is equal to or smaller than a cross-sectional area of the guide wire lumen.

14. The elongated medical body according to claim 10, wherein the shaft portion is a first shaft portion, and further comprising a second shaft portion and a third shaft portion.

15. The elongated medical body according to claim 14, wherein a total cross-sectional area of the first shaft portion, the second shaft portion and the third shaft portion is equal to or larger than a cross-sectional area of the guide wire lumen.

16. The elongated medical body according to claim 10, wherein a distal portion of the shaft portion is connected to the proximal end side of the main body portion at the connection portion, the distal portion of the shaft portion having a tapering shape.

17. The elongated medical body according to claim 10, wherein the shaft portion is a first shaft portion, and further comprising a second shaft portion positioned adjacent to and extending along the first shaft portion, the second shaft portion having a distal end that is spaced proximally from the proximal end side of the main body portion and the connection portion.

18. An elongated medical body in combination with a guide wire, the elongated medical body being usable together with a catheter having a lumen so that a distal end of the elongated medical body is positionable in the lumen of the catheter to assist insertion of the catheter into a living body,
the guide wire being an elongated guide wire that has an outer diameter equal to or greater than 0.035 in;
the elongated medical body comprising:
a main body portion extending in an axial direction between a proximal end of the main body portion and a distal end of the main body portion, the main body portion including a guide wire lumen extending axially throughout the main body portion from the proximal end of the main body portion to the distal end of the main body portion, the guide wire lumen being open to outside the main body portion at both the proximal end of the main body portion and the distal end of the main body so that the guide wire is positionable in the guide wire lumen while also extending distally beyond the distal end of the main body and proximally beyond the proximal end of the main body portion, the guide wire lumen possessing an inner diameter;
a shaft portion extending in the axial direction, the shaft portion having a distal portion fixed to main body portion at a connection portion while an adjacent part of the shaft portion extends away from the main body portion, the shaft portion possessing an outer diameter;
a guide wire port through which the guide wire enters and exits the guide wire lumen in the main body portion, the guide wire port being provided at the connection portion between the main body portion and the shaft portion; and
the inner diameter of the guide wire lumen being equal to or larger than the outer diameter of the shaft portion.

19. The elongated medical body in combination with the guide wire according to claim 18, wherein the shaft portion is a first shaft portion, and further comprising at least one additional shaft portion extending along the first shaft portion in the axial direction, the first shaft portion and the at least one additional shaft portion constituting all of the shaft portions of the elongated medical device, a total cross-sectional area of the all of the shaft portions being equal to or larger than a cross-sectional area of the guide wire lumen.

20. The elongated medical body in combination with the guide wire according claim 18, wherein the main body portion is made of resin and the shaft portion is made of metal.
